(19) United States
(12) Patent Application Publication
(10) Pub. No.: US 2021/0291131 A1
(43) Pub. Date: Sep. 23, 2021

(54) FACIAL PACK MANUFACTURING APPARATUS CAPABLE OF DISCHARGING MIXTURE WITHOUT REMNANT

(71) Applicant: Minho SEO, Bucheon-si (KR)

(72) Inventor: Minho SEO, Bucheon-si (KR)

(21) Appl. No.: 17/257,830

(22) PCT Filed: Jul. 3, 2019
(86) PCT No.: PCT/KR2019/008090

§ 371 Date: Jan. 4, 2021

(30) Foreign Application Priority Data
Jul. 5, 2018 (KR) 10-2018-0078217

(51) Int. Cl. B01F 15/02; B01F 7/18; B01F 7/00; A45D 34/04

(52) U.S. Cl. CPC B01F 15/0293; B01F 7/18; B01F 2215/0031; B01F 15/0279; A45D 34/04; B01F 7/00291

(57) ABSTRACT

A facial pack manufacturing apparatus capable of discharging a mixture without a remnant, according to the present invention, comprises a main body formed with an injection hole into which a raw material of a facial pack and a solid agent are injected; a mixing unit for mixing the raw material and the solid agent introduced into the injection hole of the main body, and discharging the mixture; and a pack manufacturing unit for receiving the mixture discharged from the mixing unit and manufacturing the mixture into a facial pack, wherein the mixing unit includes: an upper body including a blade protruding downward so as to be rotatable; a lower body coupled to a lower portion of the upper body so that an upper side thereof is opened to insert the blade, and having a discharge unit through which the mixture is discharged and a through hole passing through the discharge unit; and an opening and closing unit, provided in the through hole so as to be vertically movable, for opening and closing the discharge unit.

### TECHNICAL FIELD

The present invention relates to a facial pack manufacturing apparatus capable of discharging a mixture without a remnant.

### BACKGROUND ART

Currently, mask packs have been used to increase moisturizing effect on dry skin or skin diseases regardless of sex.

In particular, in recent years, men as well as women are increasingly interested in skin care. As interest in skin care increases, mask packs that facilitate skin care have been widely used.

Conventionally, a commercially available mask pack is a product made by attaching and bonding materials to a nonwoven fabric, and is distributed while being hygienically packaged.

However, a conventional mask pack has a shelf life and may be deteriorated or contaminated with bacteria during the distribution process.

In addition, there is no way for users to check for contamination, and sometimes users may use contaminated packs to cause side effects on the skin.

Furthermore, in order to solve the above-described problem, some users directly grind the material and paste it onto a nonwoven fabric to produce a mask pack.

However, the process of making the material is also complicated, and it was difficult to attach the material to a nonwoven fabric, so that it is very inconvenient for the user to make and use the mask pack himself.

In addition, even if ingredients such as cucumbers, carrots, tomatoes, and kiwi are used to enhance the beauty effect, they are simply ground and pasted on the face, and there is difficulty in use due to discomfort such as flowing down from the face.

In addition, since the nonwoven fabric absorbs other moisture when moisture evaporates, it is discomfort to remove the nonwoven fabric after a certain period of time when the nonwoven fabric is attached to the skin, and side effects occur if the time to remove it is not kept.

In particular, in the case of a mask pack that directly adheres to the skin for beauty, unverified products other than those that have been certified by the Food and Drug Administration or the Ministry of Food Safety may be used, and thus, means to prevent this has been required.

There has been developed a manufacturing device for manufacturing a mask pack due to this request. However, the pack manufacturing device manufactures a mask pack by introducing a material and a solid agent that hardens the material. Thereafter, the material and the solid material are discharged through a pipe, and in the process of discharging the solid material, a part of the material remain in the pipe.

In particular, a solenoid valve is used to discharge through a pipe. When a solenoid valve is used, a residual amount remains in the pipe.

As described above, when the remaining amount remains in the pipe, the solid agent may be hardened and the pipe may be clogged. And to manage this, after separating the solenoid valve and the pipe from each other, the pipe must be cleaned separately.

In addition, when another material is used while the remaining amount is left in the pipe, the material is mixed with the previously used material so that and there is a problem in use.

### DOCUMENT OF RELATED ART

### Patent Document

(Patent document 1) Korean Registered Patent No. 10-1548510

(Patent document 2) Korean Unexamined Patent Publication No. 10-2010-0137219

(Patent document 3) Korean Registered Patent No. 10-1846686

### DISCLOSURE

### Technical Problem

To solve the problems described above, an object of the present invention is to provide a facial pack manufacturing apparatus capable of discharging a mixture without a remnant, which includes a discharge unit for discharging a mixture of a raw material and a solid agent, which is formed vertically on a lower body to allow an opening and closing unit to be opened or closed while moving vertically, such that, when the opening and closing unit moves upward, the discharge unit is opened to allow the mixture to be quickly discharged without leaving the mixture in the lower body through a discharge hole formed vertically. Accordingly, since no mixture remains in the lower body and the discharge hole, the facial pack manufacturing apparatus may be used without clogging the lower body and the discharge hole.

In addition, another object of the present invention is to provide a facial pack manufacturing apparatus capable of discharging a mixture without a remnant, which is capable of rapidly discharging a mixture through a discharge hole when an opening and closing unit moves upward to open a discharge hole, and at the same time, completely discharging the mixture, thereby manufacturing another mask pack in a state that a material is not mixed with other raw materials.

### Technical Solution

To achieve the objects, according to the present invention, there is provided a facial pack manufacturing apparatus capable of discharging a mixture without a remnant, which includes a main body formed with an injection hole into which a raw material of a facial pack and a solid agent are injected; a mixing unit for mixing the raw material and the solid agent introduced into the injection hole of the main body, and discharging the mixture; and a pack manufacturing unit for receiving the mixture discharged from the mixing unit and manufacturing the mixture into the facial pack, wherein the mixing unit includes: an upper body including a blade protruding downward so as to be rotatable; a lower body coupled to a lower portion of the upper body, and having a discharge unit through which the mixture is discharged and a through hole formed through the discharge unit, in which an upper side of the lower body is opened so that the blade is inserted into the lower body; and an opening and closing unit provided in the through hole and vertically movable therein for opening and closing the discharge unit.

In addition, the discharge unit includes a protrusion defined by an inner lower surface of the lower body that protrudes downward, in which the protrusion has a cross-sectional area that gradually becomes small in a downward direction and is formed with a through hole vertically formed through the protrusion; and a discharge pipe extending downward from the protrusion around the through hole and having a discharge hole through which the mixture is discharged, in which a lower end of the discharge pipe communicates with an inner portion of the lower body.

In addition, the opening and closing unit includes a vertical movement shaft coupled to the through hole to move vertically; a sealing unit provided inside the protrusion to be tightly closed to an inner circumferential surface of the protrusion, and coupled to an upper end of the vertical movement shaft; and an elastic member having both ends supported on a lower portion of the vertical movement shaft and a lower side surface of the protrusion, and applying an elastic force in a vertical direction to allow the sealing unit to close an upper end of the discharge hole.

In addition, the discharge unit further includes a locking unit protruding from an inner lower side surface of the discharge unit such that an upper end of the through hole is located at a position higher than a position of the upper end of the discharge hole, the discharge pipe protrudes to allow a lower end of the discharge hole to be located at a position lower than a position of a lower end of the through hole, and the mixture is introduced into and discharged through the discharge hole, in which the discharge hole is configured to be longer than a vertical length of the through hole, and has an upper end lower than an upper end of the through hole and a lower end positioned below a lower end of the through hole when the sealing unit is moved upward so that the discharge hole is opened.

In addition, the mixing unit further includes a rotating unit provided in a lower portion of the lower body and rotating a rotation shaft in one direction and then rotating the rotation shaft in an opposite direction; and a pressing unit coupled to the rotation shaft to press a lower end of the vertical movement shaft when rotating in one direction, thereby moving the vertical movement shaft and the sealing unit upward, wherein the elastic member is compressed when the vertical movement shaft is pressed and expands when the rotation shaft is rotated in the opposite direction, thereby returning the vertical movement shaft and the sealing unit moved upward to original positions.

In addition, the vertical movement shaft has a support portion protruding from a lower portion toward an outer circumferential surface of the vertical movement shaft to support a lower end of the elastic member, and a locking groove is recessed in an upper circumferential surface of the vertical movement shaft, and a head portion is formed on an upper portion of the vertical movement shaft, wherein the sealing unit has a coupling hole into which the head portion of the vertical movement shaft is press-fitted, and a locking sill protruding from an inner circumferential surface of the coupling hole, in which the locking sill is inserted into the locking groove and a lower end of the head portion is supported on the locking sill.

In addition, the sealing unit includes a concaved groove which is concaved in a lower side surface of the sealing unit and into which the locking unit is inserted, and a contact protrusion protruding from the lower side surface of the sealing unit to surround the locking unit and in close contact with an inner lower side surface of the lower body, wherein the contact protrusion is compressed while the elastic member is expanded and is in close contact with the inner lower side of the lower body to block the mixture from flowing into the concaved groove.

### Advantageous Effects

As described above, according to the present invention, the facial pack manufacturing apparatus capable of discharging a mixture without a remnant includes a discharge unit for discharging a mixture of a raw material and a solid agent, which is formed vertically on a lower body to allow an opening and closing unit to be opened or closed while moving vertically, such that, when the opening and closing unit moves upward, the discharge unit is opened to allow the mixture to be quickly discharged without leaving the mixture in the lower body through a discharge hole formed vertically. Accordingly, since no mixture remains in the lower body and the discharge hole, the facial pack manufacturing apparatus may be used without clogging the lower body and the discharge hole.

In addition, the facial pack manufacturing apparatus capable of discharging a mixture without a remnant is capable of rapidly discharging a mixture through a discharge hole when an opening and closing unit moves upward to open a discharge hole, and at the same time, completely discharging the mixture, thereby manufacturing another mask pack in a state that the material is not mixed with another raw material.

### DESCRIPTION OF DRAWINGS

FIG. 1 is a perspective view showing a facial pack manufacturing apparatus capable of discharging a mixture without a remnant according to a preferred embodiment of the present invention.

FIG. 2 is an exploded perspective view showing a facial pack manufacturing apparatus capable of discharging a mixture without a remnant according to a preferred embodiment of the present invention.

FIG. 3 is a side view showing a mixing unit of a facial pack manufacturing apparatus capable of discharging a mixture without a remnant according to a preferred embodiment of the present invention.

FIG. 4 is an exploded perspective view showing a mixing unit of a facial pack manufacturing apparatus capable of discharging a mixture without a remnant according to a preferred embodiment of the present invention.

FIG. 5 is an exploded perspective view showing a sealing unit of a facial pack manufacturing apparatus capable of discharging a mixture without a remnant according to a preferred embodiment of the present invention.

FIG. 6 is a cross-sectional view showing a sealing unit of a facial pack manufacturing apparatus capable of discharging a mixture without a remnant according to a preferred embodiment of the present invention.

FIGS. 7 and 8 are cross-sectional views taken along line A-A′ of FIG. 3.

### BEST MODE

### Mode for Invention

Hereinafter, a facial pack manufacturing apparatus capable of discharging a mixture without a remnant according to an embodiment of the present invention will be described with reference to drawings.

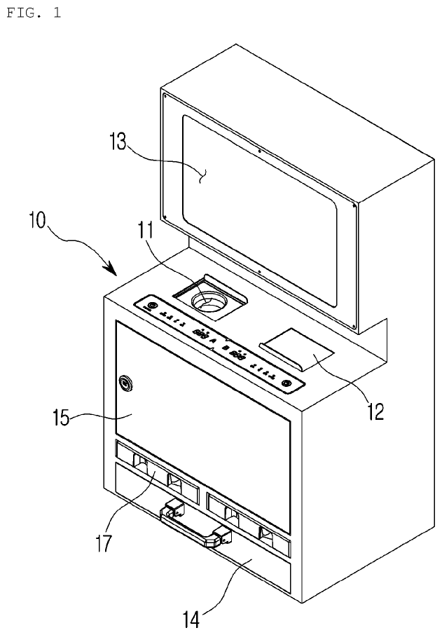

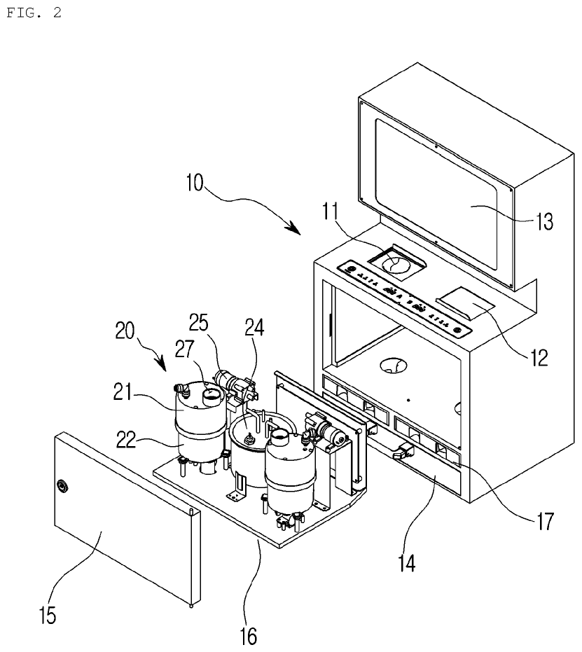

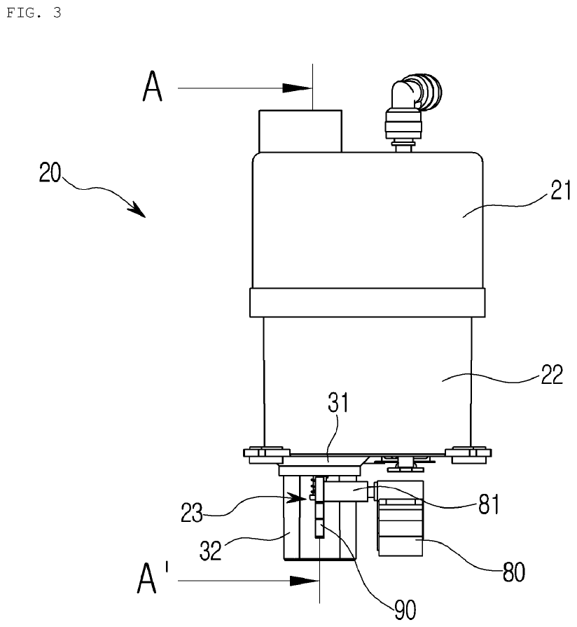

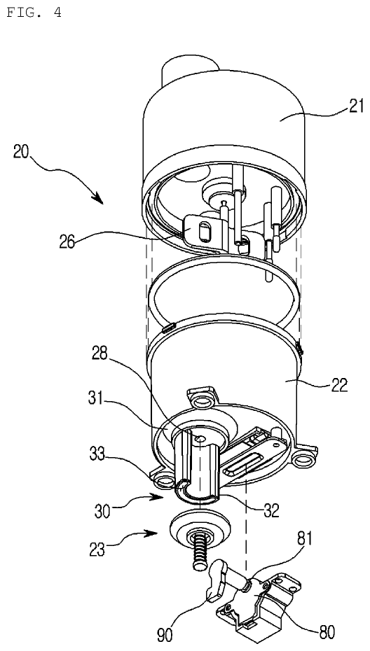

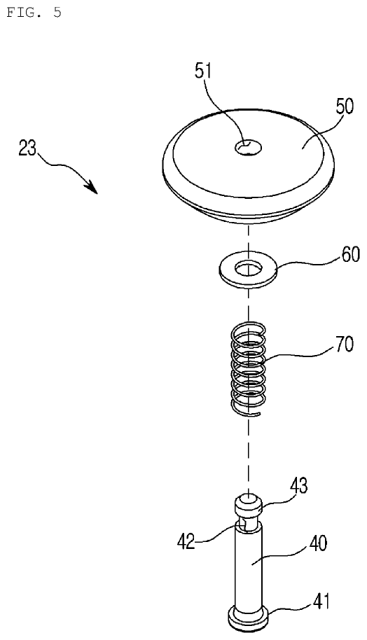

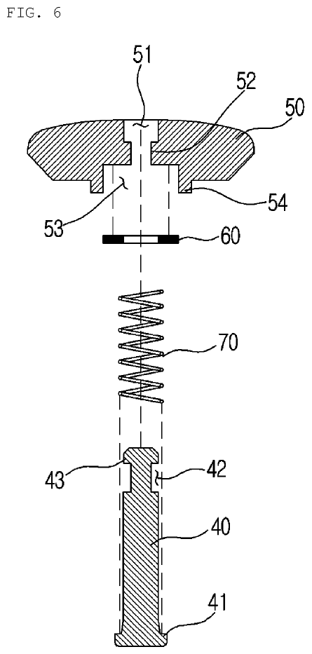

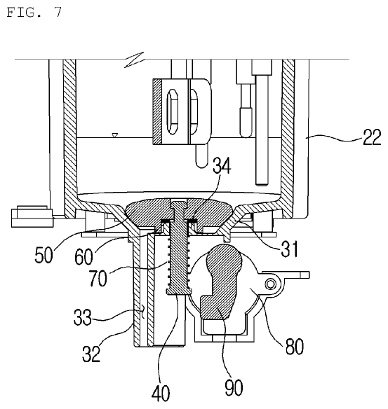

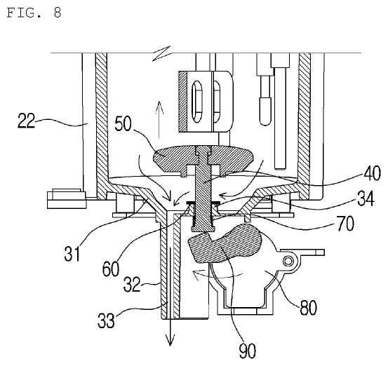

FIG. 1 is a perspective view showing a facial pack manufacturing apparatus capable of discharging a mixture without a remnant according to a preferred embodiment of the present invention. FIG. 2 is an exploded perspective view showing a facial pack manufacturing apparatus capable of discharging a mixture without a remnant according to a preferred embodiment of the present invention. FIG. 3 is a side view showing a mixing unit of a facial pack manufacturing apparatus capable of discharging a mixture without a remnant according to a preferred embodiment of the present invention. FIG. 4 is an exploded perspective view showing a mixing unit of a facial pack manufacturing apparatus capable of discharging a mixture without a remnant according to a preferred embodiment of the present invention. FIG. 5 is an exploded perspective view showing a sealing unit of a facial pack manufacturing apparatus capable of discharging a mixture without a remnant according to a preferred embodiment of the present invention. FIG. 6 is a cross-sectional view showing a sealing unit of a facial pack manufacturing apparatus capable of discharging a mixture without a remnant according to a preferred embodiment of the present invention. FIGS. 7 and 8 are cross-sectional views taken along line A-A′ of FIG. 3.

Referring to FIGS. 1 to 2, a facial pack manufacturing apparatus capable of discharging a mixture without a remnant according to the present invention includes a main body 10, a mixing unit 20, and a pack manufacturing unit 17.

First, the main body 10 is formed with an injection hole 11 into which a raw material and a solid agent of a massage pack are introduced. In this case, the injection holes 11 may be formed on the left and right sides, but the embodiment is not limited thereto.

In addition, the main body 10 is provided with an injection cover 12 capable of opening and closing the injection hole 11.

Further, an advertisement panel 13 capable of advertising is provided on an upper portion of the main body 10, and may display advertisements such as beauty, raw materials, solid agents, and cosmetics.

In addition, the main body 10 includes a power button for driving the mixing unit 20 to be described later, a status display unit indicating status such as operation, mask pack manufacturing completion, water supply, cleaning, etc., and a time display unit for displaying time.

A mixing unit 20 to be described later is provided in the middle of the main body 10.

In addition, a pack manufacturing unit 17 for manufacturing a mask pack, which will be described later and a mixture is injected from the mixing unit 20 thereto, is provided below the middle portion of the main body 10.

In addition, a water collection unit 14 for collecting water discharged after being injected into the mixing unit 20 for cleaning after the mask pack is manufactured is provided at the lowermost portion of the main body 10.

The mixing unit 20 mixes the raw material and the solid agent injected into the injection hole 11 of the main body 10, and discharges the mixture.

In this case, the main body 10 is provided with a door 15 for opening the middle portion to facilitate cleaning after the mixing unit 20 is separated.

Further, the mixing unit 20 is provided on the upper portion of a plate 16 provided to slide back and forth through the opened door 15.

In addition, the mixing unit 20 is provided on the plate 16 to be detachably attached thereto.

The mixture discharged from the mixing unit 20 is provided to the pack manufacturing unit 17, and the pack manufacturing unit 17 manufactures a mask pack with the mixture.

In this case, the pack manufacturing unit 17 may be provided with a mold in a preset shape, and the mixture may be injected into the mold to manufacture a mask pack in the preset shape.

Meanwhile, the above-described mixing unit 20 will be described in detail.

Referring to FIGS. 3 to 8, the mixing unit 20 includes an upper body 21, a lower body 22, an opening/closing unit 23, a water storage unit 24, and a pump unit 25.

The upper body 21 includes a blade 26 which protrudes downward and is rotatable.

In addition, a rotary motor for rotating the blade 26 is provided in the upper body 21.

In addition, the upper body 21 is formed with a connection hole 27 in communication with the injection hole 11 such that the raw material and the solid agent injected into the injection hole 11 are introduced and transferred to the lower body 22 to be described later.

The water storage unit 24 may be provided on the plate 16, and store water supplied from an outside.

The pump unit 25 supplies water stored in the water storage unit 24 to the upper body 21. In this case, the water supplied to the upper body 21 is introduced into the lower body 22 through the upper body 21.

The lower body 22 is coupled to the lower portion of the upper body 21 so that the upper side is opened and the blade 26 is inserted. In addition, the lower body 22 is formed with a discharge unit 30 through which the mixture is discharged and a through hole 28 penetrating the discharge unit 30.

In addition, the discharge unit 30 includes a protrusion 31 and a discharge pipe 32.

The protrusion 31 is defined by an inner lower surface of the lower body 22 that protrudes downward, in which the protrusion 31 has a cross-sectional area that gradually becomes small in a downward direction and is formed with a through hole 28 vertically formed through the protrusion 31.

In this case, the inner bottom surface of the lower body 22 is formed to be inclined toward the protrusion 31, so that the mixture is easily introduced into the protrusion 31.

The discharge pipe 32 extends downward from the protrusion 31 around the through hole 28 and has a discharge hole 33 through which the mixture is discharged, in which a lower end of the discharge pipe 32 communicates with an inner portion of the lower body 22. In this case, the discharge hole 33 is formed vertically.

In addition, the discharge unit 30 further includes a locking unit 34.

The locking unit 34 protrudes from the inner lower side surface of the discharge unit so that the upper end of the through hole 28 is located at a position higher than that of the upper end of the discharge hole 33.

In this case, the discharge pipe 32 protrudes to allow the lower end of the discharge hole 33 to be located at a position lower than that of the lower end of the through hole 28.

Accordingly, when the sealing unit 50 moves upward so that the discharge hole 33 is opened, the mixture is introduced into and discharged through the discharge hole 33, in which the discharge hole 33 is configured to be longer than a vertical length of the through hole 28, and has an upper end lower than an upper end of the through hole 28 and a lower end positioned below a lower end of the through hole 28.

That is, when the discharge hole 33 is opened by the sealing unit 50, the air pressure lower than that in the through hole 28 is formed in the discharge hole 33 which is longer than the through hole 28 and has an upper end lower than that of the through hole 28, so that the mixture is hardly discharged through the through hole 28.

In particular, the mixture may be discharged as quickly as possible into the discharge hole 33 without a remnant by the discharge unit 30 having the above-described structure.

The opening and closing unit 23 is provided in the through hole 28 to be movable vertically, and opens and closes the discharge unit 30.

The opening and closing unit 23 includes a vertical movement shaft 40, a sealing unit 50 and an elastic member 70.

The vertical movement shaft 40 is coupled to the through hole 28 to be movable vertically.

In addition, the vertical movement shaft 40 has a supporting portion 41 protruding from the outer circumferential surface of a lower portion to support the lower end of the elastic member 70, and is concaved on the upper outer circumferential surface to form a locking groove 42, and is formed with the head portion 43 on the upper portion thereof.

The sealing unit 50 is provided inside the protrusion 31 to be in close contact with the inner circumferential surface of the protrusion 31, and is coupled to the upper end of the vertical movement shaft 40.

In addition, the sealing unit 50 is formed with a coupling hole 51 through which the head portion 43 of the vertical movement shaft 40 is forcibly fitted, and a locking sill 52 protrudes from the inner circumferential surface of the coupling hole 51, which is inserted into the locking groove 42 and supports the lower end of the head portion 43.

In addition, the sealing unit 50 is formed with an inner inlet groove 53 of which the locking portion 34 is inserted into the lower side. In addition, the sealing unit 50 is formed with a contact protrusion 54 that protrudes from the lower side surface to surround the locking portion 34 and is in close contact with the inner lower side of the lower body 22.

The contact protrusion 54 is compressed while the elastic member 70 is expanded and is in close contact with the inner lower side surface of the lower body 22 to block the mixture from flowing into the inner inlet groove 53.

In this case, the opening and closing unit 23 is formed such that the vertical movement shaft 40 passes therethrough, and is provided with the sealing member 60 in the inner inlet groove 53 such that the mixture flowing into the inlet groove 53 is prevented from being discharged into the through hole 28.

In addition, the sealing unit 50 is coated to prevent the mixture from adhering thereon.

The elastic member 70 is provided to allow both ends thereof to be supported on the support portion 41 of the lower portion of the vertical movement shaft 40 and the lower side surface of the protrusion 31, and applies an elastic force to allow the sealing unit 50 to close the upper end of the discharge hole 33.

Meanwhile, the mixing unit 20 further includes a rotating unit 80 and a pressing unit 90.

The rotating unit 80 is provided on a lower portion of the lower body 22, and after rotating the rotating shaft 81 in one direction, rotates the rotating shaft 81 in the opposite direction.

In this case, the rotating unit 80 rotates the rotation shaft 81 in one direction, and then rotates the rotation shaft 81 in the opposite direction as long as the rotation shaft 81 is rotated in one direction to return the rotation shaft 81 to its original position.

When the pressing unit 90 is coupled to the rotation shaft 81 and presses the lower end of the vertical movement shaft 40 to move the vertical movement shaft 40 and the sealing unit 50 upward when the rotation shaft 81 is rotated in one direction.

In this case, the elastic member 70 is compressed when the vertical movement shaft 40 is pressed, expands when the rotation shaft 81 is rotated in the opposite direction, and returns the vertical movement shaft 40 and the sealing unit 50 moved upward to the original positions.

1. A facial pack manufacturing apparatus capable of discharging a mixture without a remnant, the facial pack manufacturing apparatus comprising:
a main body formed with an injection hole into which a raw material of a facial pack and a solid agent are injected;
a mixing unit for mixing the raw material and the solid agent introduced into the injection hole of the main body, and discharging the mixture; and a pack manufacturing unit for receiving the mixture discharged from the mixing unit and manufacturing the mixture into the facial pack,
wherein the mixing unit includes:
an upper body including a blade protruding downward so as to be rotatable; a lower body coupled to a lower portion of the upper body, and having a discharge unit through which the mixture is discharged and a through hole formed through the discharge unit, in which an upper side of the lower body is opened so that the blade is inserted into the lower body; and an opening and closing unit provided in the through hole and vertically movable therein for opening and closing the discharge unit.

2. The facial pack manufacturing apparatus of claim 1, wherein the discharge unit includes:
a protrusion defined by an inner lower surface of the lower body that protrudes downward, in which the protrusion has a cross-sectional area that gradually becomes small in a downward direction and is formed with a through hole vertically formed through the protrusion; and a discharge pipe extending downward from the protrusion around the through hole and having a discharge hole through which the mixture is discharged, in which a lower end of the discharge pipe communicates with an inner portion of the lower body.

3. The facial pack manufacturing apparatus of claim 2, wherein the opening and closing unit includes:
a vertical movement shaft coupled to the through hole to move vertically; a sealing unit provided inside the protrusion to be tightly closed to an inner circumferential surface of the protrusion, and coupled to an upper end of the vertical movement shaft; and an elastic member having opposite ends supported on a lower portion of the vertical movement shaft and a lower side surface of the protrusion, and applying an elastic force in a vertical direction to allow the sealing unit to close an upper end of the discharge hole.

4. The facial pack manufacturing apparatus of claim 3, wherein the discharge unit further includes a locking unit protruding from an inner lower side surface of the discharge unit such that an upper end of the through hole is located at a position higher than a position of the upper end of the discharge hole, the discharge pipe protrudes to allow a lower end of the discharge hole to be located at a position lower than a position of a lower end of the through hole, and the mixture is introduced into and discharged through the discharge hole, in which the discharge hole is configured to be longer than a vertical length of the through hole, and has an upper end lower than an upper end of the through hole and a lower end positioned below a lower end of the through hole when the sealing unit is moved upward so that the discharge hole is opened.

5. The facial pack manufacturing apparatus of claim 3, wherein the mixing unit further includes:
a rotating unit provided in a lower portion of the lower body and rotating a rotation shaft in one direction and then rotating the rotation shaft in an opposite direction; and a pressing unit coupled to the rotation shaft to press a lower end of the vertical movement shaft when rotating in one direction, thereby moving the vertical movement shaft and the sealing unit upward, and wherein the elastic member is compressed when the vertical movement shaft is pressed and expands when the rotation shaft is rotated in the opposite direction, thereby returning the vertical movement shaft and the sealing unit moved upward to original positions.

6. The facial pack manufacturing apparatus of claim 3, wherein the vertical movement shaft has a support portion protruding from a lower portion toward an outer circumferential surface of the vertical movement shaft to support a lower end of the elastic member, and a locking groove is recessed in an upper circumferential surface of the vertical movement shaft, and a head portion is formed on an upper portion of the vertical movement shaft, and wherein the sealing unit has a coupling hole into which the head portion of the vertical movement shaft is press-fitted, and a locking sill protruding from an inner circumferential surface of the coupling hole, in which the locking sill is inserted into the locking groove and a lower end of the head portion is supported on the locking sill.

7. The facial pack manufacturing apparatus of claim 4, wherein the sealing unit includes a concaved groove which is concaved in a lower side surface of the sealing unit and into which the locking unit is inserted, and a contact protrusion protruding from the lower side surface of the sealing unit to surround the locking unit and in close contact with an inner lower side surface of the lower body, and wherein the contact protrusion is compressed while the elastic member is expanded and is in close contact with the inner lower side of the lower body to block the mixture from flowing into the concaved groove.
